(19) United States
(12) Patent Application Publication
(10) Pub. No.: US 2022/0160537 A1
(43) Pub. Date: May 26, 2022

(54) DEVICES, SYSTEMS, AND METHODS FOR TREATING DISEASE USING ELECTRICAL STIMULATION

(71) Applicant: Phagenesis Limited, Manchester (GB)

(72) Inventor: Conor Mulrooney, Manchester (GB)

(21) Appl. No.: 17/455,571

(22) Filed: Nov. 18, 2021

Related U.S. Application Data
(60) Provisional application No. 63/198,939, filed on Nov. 24, 2020.

(51) Int. Cl. A61F 5/58; A61N 1/05

(52) U.S. Cl. CPC A61F 5/58; A61N 1/0551

(57) ABSTRACT

Electrical stimulation devices and associated systems and methods are disclosed herein. In some embodiments, the electrical stimulation device comprises an elongated member configured to be orally or nasally inserted into a patient's pharynx. The device may further include a conductive element carried by a distal portion of the elongated member and configured to deliver stimulation energy to nearby tissue to treat one or more of gastric motility, sleep apnea, and speech disorders.

### CROSS-REFERENCE TO RELATED APPLICATION(S)

The present application claims the benefit of priority to U.S. Provisional Patent Application No. 63/198,939, titled DEVICES, SYSTEMS, AND METHODS FOR TREATING DISEASE USING ELECTRICAL STIMULATION, filed Nov. 24, 2020, which is incorporated by reference herein in its entirety.

### TECHNICAL FIELD

The present disclosure is directed generally to devices, systems, and methods for treating disease using electrical stimulation.

### BACKGROUND

The pharynx serves multiple and diverse roles—mastication, breathing, swallowing, speaking, taste and smell, heat, humidify and filter air, and protect the airways. This single structure serves diverse and highly complex functions, many of which may not be carried out simultaneously. For example, the pharynx is a structure shared by both the respiratory and digestive pathways and acts as a mechanical “switch” to direct incoming air and solids to the appropriate anatomical systems during breathing and swallowing.

During normal respiration, structures of the pharynx assume positions that maximize the patency of the airway. As air is inhaled, tonic activation actively maintains pharyngeal position and phasic activation via the negative pressure reflex resists vacuum-induced changes in pharyngeal position. Because the pharynx is situated at the literal crossroad of the respiratory and gastrointestinal intakes, pharyngeal structural and/or postural dysfunction may result in a variety of disorders including obstructive sleep apnea, gastric motility disorders, and speech disorders. In addition to the immediate health concerns introduced by these disorders, many are associated with an increased risk of additional comorbidities such as heart attack, stroke, hypertension, diabetes, development of carotid artery atherosclerosis, pulmonary aspiration and aspiration pneumonia, among others.

Existing treatments for pharyngeal disorders suffer from limited effectiveness or compliance. Accordingly, there remains a need for improved devices and methods for treating certain conditions affected by pharyngeal dysfunction.

### SUMMARY

The present technology relates to electrical stimulation devices and associated systems and methods. In particular embodiments, the present technology comprises electrical stimulation devices configured to perform pharyngeal electrical stimulation (PES) to treat one or more conditions. The subject technology is illustrated, for example, according to various aspects described below, including with reference to FIGS. 1-3B. Various examples of aspects of the subject technology are described as numbered clauses (1, 2, 3, etc.) for convenience. These are provided as examples and do not limit the subject technology.

1. A method for rehabilitating a patient's speech, the method comprising:
via a nasal or oral cavity, positioning a conductive element of a stimulation device in a lumen of an upper gastrointestinal tract of a patient; and
delivering stimulation energy to sensory nerves proximate the lumen, the sensory nerves associated with speech.

2. The method of Clause 1, wherein delivering stimulation energy to the sensory nerves excites central nervous system regions associated with speech control and modulation.

3. The method of Clause 1 or Clause 2, wherein the sensory nerves include one or more cranial nerves.

4. The method of any one of Clauses 1 to 3, wherein the sensory nerves include the glossopharyngeal nerve.

5. The method of any one of Clauses 1 to 4, wherein the sensory nerves include the vagus nerve.

6. The method of any one of Clauses 1 to 5, wherein the sensory nerves include sensory nerves proximate one or more structure of the larynx.

7. The method of any one of Clauses 1 to 6, wherein delivering the stimulation energy improves at least one of sound formation, quality of tone, intelligibility of communication, or level of speech.

8. The method of any one of Clauses 1 to 7, wherein the stimulation energy is delivered while the conductive element is positioned within the pharynx.

9. The method of any one of Clauses 1 to 8, wherein the stimulation energy is delivered while the conductive element is positioned within the laryngopharynx.

10. The method of any one of Clauses 1 to 9, wherein the stimulation energy is delivered while the conductive element is in apposition with a posterior wall of the pharynx.

11. The method of any one of Clauses 1 to 10, wherein the stimulation energy is delivered while the conductive element is in apposition with at least one of an anterior wall of the pharynx, a lateral wall of the pharynx, or a medial wall of the pharynx.

12. The method of any one of Clauses 1 to 11, wherein the stimulation device includes a feeding tube, and wherein the feeding tube remains positioned in the patient's upper gastrointestinal tract when the stimulation energy is delivered.

13. The method of any one of Clauses 1 to 12, wherein the stimulation energy has a frequency of about 5 Hz to about 80 Hz.

14. The method of any one of Clauses 1 to 13, wherein the stimulation energy has an amplitude of about 1 mA to about 50 mA.

15. The method of any one of Clauses 1 to 14, wherein the stimulation energy has a pulse width of about 200 μS to about 1 mS.

16. A device for delivering an electrical stimulus to nerves proximate a pharynx of a human patient, the device comprising:
an elongated member having a proximal portion configured to be coupled to an energy source and a distal portion configured to be positioned at a treatment site within the pharynx; and
a conductive element carried by the distal portion of the elongated member and configured to be electrically coupled to the energy source to deliver an electrical stimulus to nerves at or proximate the pharyngeal wall at the treatment site, wherein the electrical stimulus is configured to improve the physical performance of speech related function.

17. The device of Clause 16, wherein the electrical stimulus is configured to improve at least one of speech intelligibility, tone, or level.

18. The device of Clause 16 or Clause 17, wherein the nerves are sensory nerves.

19. The device of any one of Clauses 16 to 18, wherein the nerves comprise one or more cranial nerves.

20. The device of any one of Clauses 16 to 19, wherein the nerves comprise glossopharyngeal nerves.

21. The device of any one of Clauses 16 to 20, wherein the nerves comprise vagus nerves.

22. The device of any one of Clauses 16 to 21, further comprising an elongated shaft configured to be slidably positioned through a lumen of the elongated member.

23. The device of Clause 22, further comprising a retaining structure configured to releasably engage the elongated shaft to fix a position of the elongated shaft relative to the elongated member.

24. The device of Clause 22 or Clause 23, wherein the elongated shaft has a proximal portion and a distal portion configured to be positioned within the patient's stomach, and wherein the elongated shaft is configured to deliver nutrients to the stomach.

25. A method for improving a patient's gastric motility, the method comprising:
via a nasal or oral cavity, positioning a conductive element of a stimulation device in a lumen of an upper gastrointestinal tract of a patient; and
delivering stimulation energy to sensory nerves proximate the lumen, the sensory nerves associated with gastric motility.

26. The method of Clause 25, wherein delivering stimulation energy to the sensory nerves drives organizational changes and/or excitability level changes in the brain.

27. The method of Clause 25 or Clause 26, wherein the sensory nerves include one or more cranial nerves.

28. The method of any one of Clauses 25 to 27, wherein the sensory nerves include the vagus nerve.

29. The method of any one of Clauses 25 to 28, wherein delivering the stimulation energy improves at least one of gastric emptying, contractility, or capacity.

30. The method of any one of Clauses 25 to 29, wherein the stimulation energy is delivered while the conductive element is positioned within the pharynx.

31. The method of any one of Clauses 25 to 30, wherein the stimulation energy is delivered while the conductive element is positioned within the esophagus.

32. The method of any one of Clauses 25 to 31, wherein the stimulation energy is delivered while the conductive element is positioned within the oropharynx.

33. The method of any one of Clauses 25 to 32, wherein the stimulation energy is delivered while the conductive element is positioned within the laryngopharynx.

34. The method of any one of Clauses 25 to 33, wherein the stimulation energy is delivered while the conductive element is in apposition with a posterior wall of the pharynx.

35. The method of any one of Clauses 25 to 34, wherein the stimulation energy is delivered while the conductive element is in apposition with at least one of an anterior wall of the pharynx, a lateral wall of the pharynx, or a medial wall of the pharynx.

36. The method of any one of Clauses 25 to 35, wherein the stimulation energy is delivered while the conductive element is in apposition with at least one of a posterior wall of the esophagus, an anterior wall of the esophagus, a lateral wall of the esophagus, or a medial wall of the esophagus.

37. The method of any one of Clauses 25 to 36, wherein the stimulation device includes a feeding tube, and wherein the feeding tube remains positioned in the patient's upper gastrointestinal tract when the stimulation energy is delivered.

38. The method of any one of Clauses 25 to 37, wherein the stimulation energy has a frequency of about 5 Hz to about 100 Hz.

39. The method of any one of Clauses 25 to 38, wherein the stimulation energy has an amplitude of about 1 mA to about 50 mA.

40. The method of any one of Clauses 25 to 39, wherein the stimulation energy has a pulse width of about 50 μS to about 100 μS.

41. A device for delivering an electrical stimulus to nerves proximate a pharynx of a human patient, the device comprising:
an elongated member having a proximal portion configured to be coupled to an energy source and a distal portion configured to be positioned at a treatment site within the pharynx; and
a conductive element carried by the distal portion of the elongated member and configured to be electrically coupled to the energy source to deliver an electrical stimulus to nerves at or proximate the pharyngeal wall or esophageal wall at the treatment site, wherein the electrical stimulus is configured to improve at least one of gastric emptying, contractility, or capacity such that gastric motility overall is improved.

42. The device of Clause 41, wherein the nerves are sensory nerves.

43. The device of Clause 41 or Clause 42, wherein the nerves comprise one or more cranial nerves.

44. The device of any one of Clauses 41 to 43, wherein the nerves comprise glossopharyngeal nerves.

45. The device of any one of Clauses 41 to 44, wherein the nerves comprise vagus nerves.

46. The device of any one of Clauses 41 to 45, further comprising an elongated shaft configured to be slidably positioned through a lumen of the elongated member.

47. The device of Clause 46, further comprising a retaining structure configured to releasably engage the elongated shaft to fix a position of the elongated shaft relative to the elongated member.

48. The device of Clause 46 or Clause 47, wherein the elongated shaft has a proximal portion and a distal portion configured to be positioned within the patient's stomach, and wherein the elongated shaft is configured to deliver nutrients to the stomach.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a fragmentary sagittal view of a pharynx of a human patient.

FIG. 2 depicts an electrical stimulation system in accordance with several embodiments of the present technology.

FIGS. 3A and 3B depict an electrical stimulation device transnasally inserted into a pharynx of a patient in accordance with several embodiments of the present technology.

### DETAILED DESCRIPTION

As previously mentioned, the present technology relates to electrical stimulation devices and associated systems and methods. In particular embodiments, the present technology comprises electrical stimulation devices configured to perform PES to treat one or more conditions, such as gastric motility, sleep apnea, and speech disorders.

### I. Anatomy and Physiology

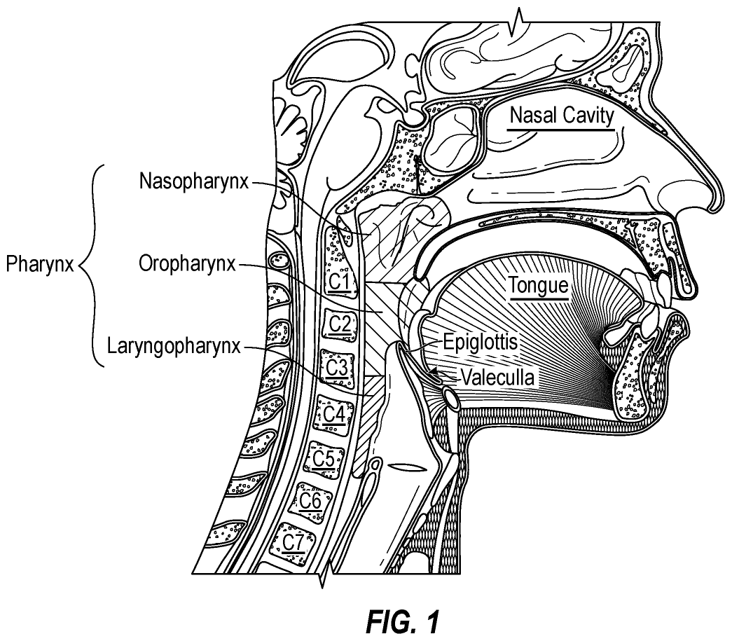

The pharynx is the part of the digestive system situated posterior to the nasal and oral cavities and posterior to the larynx. It is therefore divisible into nasal, oral, and laryngeal parts: the (1) nasopharynx, (2) oropharynx, and (3) laryngopharynx. With reference to FIG. 1, the pharynx extends from the base of the skull down to the inferior border of the cricoid cartilage (around the C6 vertebral level), where it becomes continuous with the esophagus. Its superior aspect is related to the sphenoid and occipital bones and the posterior aspect to the prevertebral fascia and muscles as well as the upper six cervical vertebrae. The pharynx is a fibromuscular tube lined by mucous membrane.

The pharynx is the common channel for deglutition (swallowing) and respiration, and the food and air pathways cross each other in the pharynx. In the anesthetized patient, the passage of air through the pharynx is facilitated by extension of the neck.

The nasopharynx, at least in its anterior part, may be regarded as the posterior portion of the nasal cavity, with which it has a common function as part of the respiratory system. The nasopharynx communicates with the oropharynx through the pharyngeal isthmus, which is bounded by the soft palate, the palatopharyngeal arches, and the posterior wall of the pharynx. The isthmus is closed by muscular action during swallowing. The choanae are the junction between nasopharynx and the nasal cavity proper.

A mass of lymphoid tissue, the nasopharyngeal tonsil is embedded in the mucous membrane of the posterior wall of the nasopharynx. Enlarged nasopharyngeal tonsils are termed “adenoids” and may cause respiratory obstruction. Higher up, a minute pharyngeal hypophysis (resembling the adenohypophysis) may be found.

The oropharynx extends inferiorward from the soft palate to the superior border of the epiglottis. It communicates anteriorly with the oral cavity by the faucial (oropharyngeal) isthmus, which is bounded superiorly by the soft palate, laterally by the palatoglossal arches, and inferiorly by the tongue. This area is characterized by a lymphatic ring composed of the nasopharyngeal, tubal, palatine, and lingual tonsils.

The mucous membrane of the epiglottis is reflected onto the base of the tongue and onto the lateral wall of the pharynx. The space on each side of the median glosso-epiglottic fold is termed the epiglottic vallecula.

The laryngopharynx extends from the superior border of the epiglottis to the inferior border of the cricoid cartilage, where it becomes continuous with the esophagus. Its anterior aspect has the inlet of the larynx and the posterior aspects of the arytenoid and cricoid cartilages. The pyriform sinus, in which foreign bodies may become lodged, is the part of the cavity of the laryngopharynx situated on each side of the inlet of the larynx.

A. Muscles

The pharynx consists of four coats of muscles, from within outward: (1) a mucous membrane continuous with that of the auditory tubes and the nasal, oral, and laryngeal cavities; (2) a fibrous coat, that is thickest in its superior extent (pharyngobasilar fascia) and that forms a median raphe posteriorly; (3) a muscular coat, described below; and (4) a fascial coat (buccopharyngeal fascia) covering the outer surface of the muscles.

The wall of the pharynx is composed mainly of two layers of skeletal muscles. The external, circular layer comprises three constrictors. The internal, chiefly longitudinal layer consists of two levators: the stylopharyngeus and the palatopharyngeus.

The chief action in which the muscles of the pharynx combine is deglutition (or swallowing), a complicated, neuromuscular act whereby food is transferred from (1) the mouth through (2) the pharynx and (3) the esophagus to the stomach. The pharyngeal stage is the most rapid and most complex phase of deglutition. During swallowing, the nasopharynx and vestibule of the larynx are sealed but the epiglottis adopts a variable position. Food is usually deviated laterally by the epiglottis and ary-epiglottic folds into the piriform recesses of the laryngopharynx, lateral to the larynx. The pharyngeal ridge is an elevation or bar on the posterior wall of the pharynx inferior to the level of the soft palate; it is produced during swallowing by transverse muscle fibers.

B. Innervation and Blood Supply

The motor and most of the sensory supply to the pharynx is by way of the pharyngeal plexus, which is formed by the pharyngeal branches of the vagus and glossopharyngeal nerves and also by sympathetic nerve fibers. The motor fibers in the plexus are carried by the vagus (although they likely represent cranial accessory nerve components) and supply all the muscles of the pharynx and soft palate except the stylopharyngeus (supplied by cranial nerve IX) and tensor veli palatini (supplied by cranial nerve V). The sensory fibers in the plexus are from the glossopharyngeal nerve, and they supply the greater portion of all three parts of the pharynx. The pharynx is supplied by branches of the external carotid (ascending pharyngeal) and subclavian (inferior thyroid) arteries.

### II. System Overview

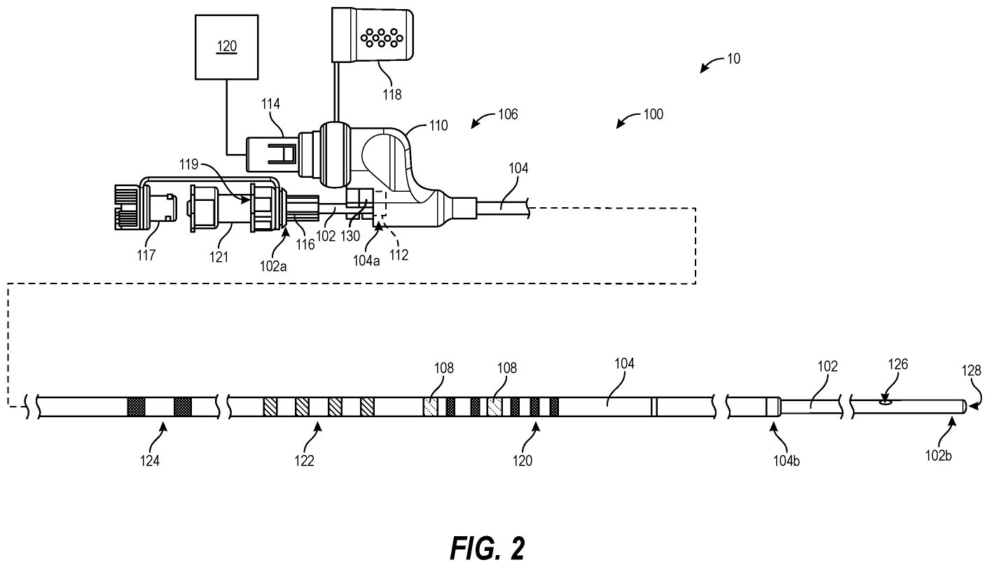

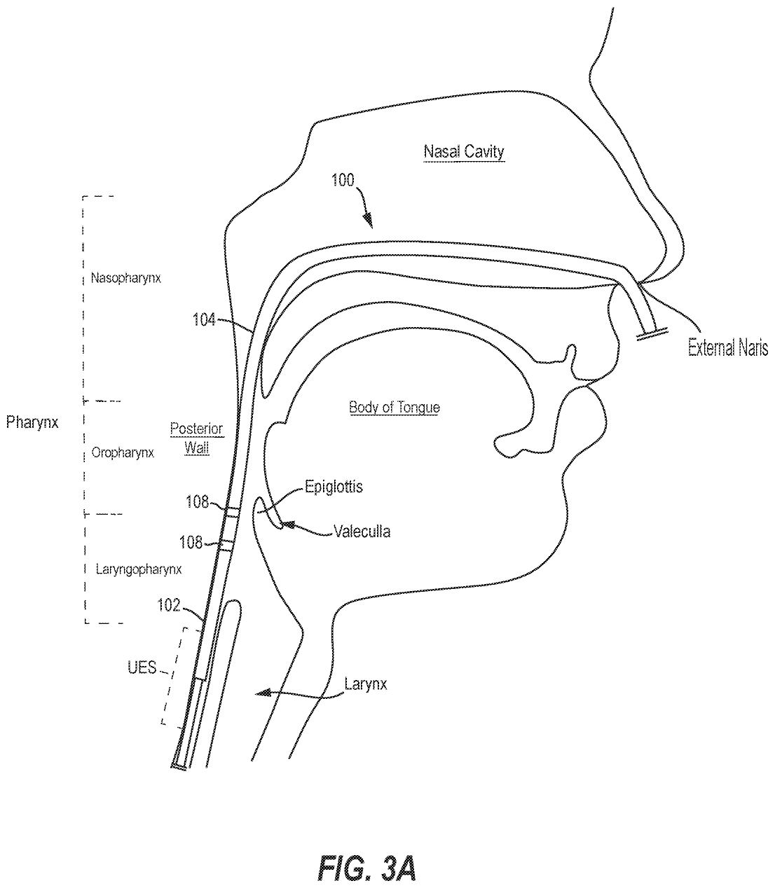

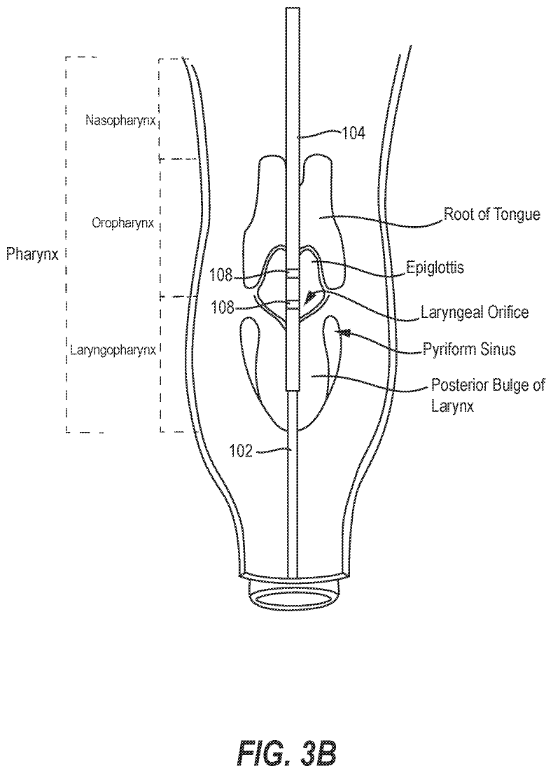

FIG. 2 depicts a treatment system 10 configured in accordance with several embodiments of the present technology. The system 10 may comprise a device 100 configured to provide intraluminal electrical stimulation to a patient suffering from a medical condition, and a current generator 120 configured to be electrically coupled to the device 100. The device 100 can include a handle assembly 106, a first elongated shaft 102 (or “first shaft 102”), and a second elongated shaft 104 (or “second shaft 104”) configured to slidably receive the first shaft 102 therethrough. The first shaft 102 can be a flexible tube configured to deliver nutrients to the patient. For example, in some embodiments the first shaft 102 comprises a nasogastric feeding tube. Each of the first and second shafts 102, 104 have a proximal portion 102a and 104a, respectively, coupled to the handle assembly 106, and a distal portion 102b and 104b, respectively, configured to be positioned within an upper gastrointestinal tract of the patient. As depicted in FIGS. 3A and 3B, when the device 100 is inserted into the patient, the distal portion 102b of the first shaft 102 is configured to be positioned within the patient's stomach, and the distal portion 104b of the second shaft 104 is configured to be positioned at a treatment site within the patient's pharynx. The device 100 further includes one or more conductive elements 108 carried by the distal portion 104b of the second shaft 104 and configured to deliver stimulation energy to nerves proximate the treatment site.

As shown in FIG. 2, the handle assembly 106 can include a hub 110 and one or more connectors and/or accessory components configured to be removably coupled to the hub 110. The hub 110 can include a housing having a first portion fixedly coupled to the proximal portion 104a of the second shaft 104, and a second portion configured to house one or more electrical components. In some embodiments, the second portion of the housing is positioned laterally of the first portion of the housing. The hub 110 may further include an electrical connector 114 at the second portion that provides an electrical interface between the second shaft 104 and the current generator 120.

In some embodiments, the device 100 includes a connector 116 coupled to the proximal portion 102a of the first shaft 102 and having one or more ports, such as port 119, configured to be coupled to one or more accessory devices or systems. For example, the port 119 may be configured to be releasably coupled to an enteral feeding set (not shown) for delivering nutrients through the first elongated shaft 102 into the patient's stomach. Additionally or alternatively, the port 119 can be configured to be releasably coupled to a guidewire assembly. For example, the port 119 can be configured to receive a guidewire (not visible) therethrough to assist with inserting the device 100 into the patient. The guidewire assembly can include a guidewire grip 121 coupled to the proximal end portion of the guidewire. In some embodiments, the connector 116 includes one or more additional ports, such as a port configured to be fluidly coupled to a syringe or other fluid source and/or pressure source. The connector 116 may further comprise a cap 117 tethered to the connector 116 and configured to be secured over the port 119 when not in use.

As previously mentioned, the first shaft 102 is configured to be inserted through a lumen of the second shaft 104. In use, the distal portion 102b of the first shaft 102 can be inserted into an opening at the proximal portion 104a of the second shaft 104 that is fixed to the hub 110. In some embodiments, the device 100 includes a sealing member (not visible) at the hub 110 that engages the first and second shafts 102, 104 to prevent fluid from within a patient being drawn up within a space between the first and second shafts 102, 104 by way of capillary action when the second shaft 104 is removed from the patient. The sealing member may also be configured to clean any matter off of the first shaft 102 as it is withdrawn from the patient.

The first shaft 102 can have an atraumatic distal tip for patient comfort and ease of inserting the first shaft 102 into the patient. The first shaft 102 can have an opening 128 at its distal end and/or one or more apertures 126 extending through the sidewall along the distal portion 102b of the first shaft 102. Nutrients can be dispersed from the first shaft 102 into the patient's stomach via the one or more apertures 126 and/or the opening 128.

Each of the conductive elements 108 may comprise an electrode, an exposed portion of a conductive material, a printed conductive material, and other suitable forms. In some embodiments, for example as shown in FIG. 1, the conductive elements 108 comprise a pair of ring electrodes configured to deliver bipolar stimulation energy. The conductive elements 108 can be crimped, welded, or otherwise adhered to an outer surface of the second shaft 104. In some embodiments, the conductive elements 108 comprise a flexible conductive material disposed on the second shaft 104 via printing, thin film deposition, or other suitable techniques. While the device 100 shown in FIG. 1 includes two conductive elements 108, in some embodiments the device 100 may include more or fewer than two conductive elements 108. For example, the device 100 may comprise a single conductive element 108 configured to generate a monopolar electric field. Such embodiments include a neutral or dispersive electrode electrically connected to the current generator 120 and positioned on the patient's skin. In some embodiments, the device 100 may include three or more conductive elements 108 spaced apart along a longitudinal axis of the second shaft 104.

The device 100 may include one or more conductive leads (not visible) extending between a proximal portion of the device 100, such as the hub 110, and the conductive elements 108. In some embodiments, for example, the conductive leads comprise two wires, each extending distally from the hub 110 through a channel (the same channel or different channels) in the second shaft 104 to a corresponding one of the conductive elements 108. The channel(s), for example, can extend longitudinally within a sidewall of the shaft 104. The conductive leads can be insulated along all or a portion of their respective lengths.

In some embodiments, the device 100 is configured such that a position of the second shaft 104 can be fixed relative to a position of the first shaft 102 (or vice versa). The position of the first shaft 102 relative to the second shaft 104 may be adjusted prior to insertion of the device 100 into the patient. Once adjustment is complete, the relative positions of the first shaft 102 and second shaft 104 may be substantially fixed. For example, as shown in FIG. 1, the proximal portion 100a of the device 100 can include a retaining structure 130 configured to be coupled to the hub 110 and/or the proximal portions 102a, 104a of one or both of the second shaft 104 and the first shaft 102. The retaining structure 130 can have a first portion on or through which the second shaft 104 and/or first shaft 102 may be movably or fixedly positioned, and a second portion moveable relative to the first portion. When the second portion is in an open position (as shown in FIG. 1), the first shaft 102 and the second shaft 104 can move longitudinally relative to one another. When the second portion is in a closed position (not shown), the longitudinal and/or radial positions of the first shaft 102 and the second shaft 104 are substantially fixed relative to one another.

The retaining structure 130 may be fixed to one of the first shaft 102 or the second shaft 104 and, at least in the open configuration, allow movement of the other of the first shaft 102 and the second shaft 104. In some embodiments, for example as shown in FIG. 1, the proximal portion of the second shaft 104 is fixed to the retaining structure 130 and the proximal portion of the first shaft 102 is slidably received by the first portion when the device 100 is assembled and the second portion is in an open position. When the second portion is in a closed position, the second portion engages the first shaft 102 and fixes the first shaft 102 relative to the second shaft 104, the hub 110, and/or the retaining structure 130.

In any case, the portion of the retaining structure 130 configured to engage the first and/or second shafts 102, 104 can comprise a high friction thermoplastic elastomer liner that engages the proximal portion of the first shaft 102 when the second portion is in the closed position. The liner can be configured to reduce the compressive force required to fix the first shaft 102 and thereby prevent pinching of the first shaft 102. Other suitable shapes, materials, positions, and configurations for the retaining structure 130 are possible. For example, the retaining structure 130 can comprise one or more magnets, a screw and threaded insert, a radial compression clip, and/or others to fix the proximal portion of the first shaft 102 to the retaining structure 130, the hub 110, and/or the second shaft 104.

As previously mentioned, the proximal portion of the device 100 and/or second shaft 104 is configured to be electrically coupled to a current generator 120 for delivering electric current to the conductive elements 108. The current generator 120, for example, can include a power source and a controller. The controller includes a processor coupled to a memory that stores instructions (e.g., in the form of software, code or program instructions executable by the processor or controller) for causing the power source to deliver electric current according to certain parameters provided by the software, code, etc. The power source of the current generator 120 may include a direct current power supply, an alternating current power supply, and/or a power supply switchable between a direct current and an alternating current. The current generator 120 can include a suitable controller that can be used to control various parameters of the energy output by the power source or generator, such as intensity, amplitude, duration, frequency, duty cycle, and polarity. Instead of or in addition to a controller, the current generator can include drive circuitry. In such embodiments, the current generator can include hardwired circuit elements to provide the desired waveform delivery rather than a software-based generator. The drive circuitry can include, for example, analog circuit elements (e.g., resistors, diodes, switches, etc.) that are configured to cause the power source to deliver electric current according to the desired parameters. For example, the drive circuitry can be configured to cause the power source to deliver periodic waveforms. In some embodiments, the drive circuitry can be configured to cause the power source to deliver a unipolar square wave.

The current generator 120 may be configured to provide a stimulation energy to the conductive elements 108 that has an intensity, amplitude, duration, frequency, duty cycle, and/or polarity such that the conductive elements 108 apply an electric field at the treatment site that promotes neuroplasticity in the areas of the brain associated with speech and/or gastric motility control. Without being bound by theory, it is believed that the treatment energy of the present technology induces and accelerates a cortical reorganization process whereby responsibility for the control and coordination of speech and/or gastric motility activity is moved from the damaged area of the brain to a complementary area of the cortical centers with intact function. The treatment energy of the present technology may also increase local levels of speech-related or gastric motility-related neurotransmitters in the pharynx and/or the esophagus.

The controller can automatically vary the voltage (to a maximum of 250V) in order to deliver the set current. In some embodiments, the only user adjustable parameter is the stimulation intensity which is derived during treatment level optimization prior to every treatment. Patient specific threshold levels are determined by establishing a sensory threshold followed by measurement of the maximum tolerated stimulation intensity. The controller may automatically calculate the correct stimulation level from the sensory threshold and maximum tolerated stimulation intensity and sets this as the output. The current generator 120 can provide, for example, a current of about 1 mA to about 50 mA, about 1 mA to about 40 mA, about 1 mA to about 30 mA, about 1 mA to about 20 mA, or about 1 mA to about 10 mA, at a frequency of about 1 Hz to about 50 Hz, about 1 Hz to about 40 Hz, about 1 Hz to about 30 Hz, about 1 Hz to about 20 Hz, about 1 Hz to about 10 Hz, about 2 Hz to about 8 Hz, about 1 Hz, about 2 Hz, about 3 Hz, about 4 Hz, about 5 Hz, about 6 Hz, about 7 Hz, about 8 Hz, about 9 Hz, or about 10 Hz, and having a pulse width of about 150 μS to about 250 μS about 175 μS to about 225 μS or about 200 μS.

The current generator 120 may also be configured to monitor contact quality between the conductive elements 108 and patient tissue during treatment set up/optimization and throughout the treatment process. In some embodiments, the current generator 120 records and stores patient information and includes a USB port to enable downloading of patient data. The current generator may include a touch screen user interface and software to guide a user through the treatment process.

### Example Methods of Use

Without being bound by theory, it is believed that increasing peripheral (sensory) feedback to higher brain centers improves the local sensory performance. Many processes in the body rely on a combination of local (peripheral) and centralized neurological control and feedback to allow multiple nerves and muscles to combine and coordinate correctly to execute a functional activity. Examples of such processes include swallowing and speech, as well as processes such as urinary/fecal control and gastric motility. Afferent nerve signals from peripheral nerves provide contextual information about the activity in question. This information is processed centrally and the outputs are efferent signals that then modulate the activity of muscles—modifying duration and intensity, changing shape and flexibility etc. After brain injury or as a consequence of persistent disuse, areas in the brain responsible for coordination or control can become dysfunctional. The absence of ‘neurological oversight’ associated with such dysfunction means that although local nerves and muscles are not damaged, they are no longer capable in isolation of delivering correct functional performance. When applied therapeutically, electrical stimulation at appropriate frequencies and intensities and locations can result in afferent nerve signals from peripheral sensory receptors. The stimulation conditions used and the afferent signals arising are designed to target and maximally excite specific areas in the brain associated with known functional processes (e.g., the swallow motor cortex). The excitation is associated with a neuroplastic functional reorganization that can restore central processing capability. As such, the device 100 of the present technology is configured to stimulate sensory nerves associated with speech and/or gastric motility, thereby inducing and accelerating a cortical reorganization process whereby responsibility for the control and coordination of speech and/or gastric motility activity is moved from the damaged area of the brain to a complementary area of the cortical centers of the brain with intact function. It is also believed that the stimulation energy of the present technology can increase local levels of speech and/or gastric motility-related neurotransmitters in the pharynx and/or the esophagus.

FIGS. 3A and 3B are a fragmentary sagittal view and an open posterior view, respectively, of a patient's throat with a device 100 of the present technology inserted through the patient's nasal cavity into the pharynx. As shown, during a given treatment, the distal portion of the second shaft 104 is positioned at a treatment site in the pharynx with the conductive elements 108 in apposition with the posterior pharyngeal wall. In some embodiments, the conductive elements 108 are positioned within a treatment window along the oropharynx and/or the laryngopharynx. In some embodiments, for example, the conductive elements 108 are positioned at a location that is equivalent to the junction between the C3 and C4 cervical vertebrae. Accordingly, when energy is delivered to the conductive elements 108 by the current generator 120, the conductive elements 108 emit an electric field that flows through the posterior wall of the pharynx, the base of the tongue, the epiglottis, and the region above the larynx. Each of these regions includes a high density of targeted sensory nerves, including the pharyngeal plexus, the superior laryngeal branch of the vagus nerve, the lingual branch of the glossopharyngeal nerve, the internal branch of the superior laryngeal nerve, and/or the external branch of the superior laryngeal nerve.

In some embodiments, the first and/or second shafts 102, 104 can comprise one or more indicators (such as indicators 120, 122, and 124 in FIG. 1) configured to facilitate insertion and positioning of the device 100 within the patient. For example, the indicator can comprise one or more visual markings that, when viewed through the patient's oral cavity, indicate the conductive elements 108 are properly positioned or that the second shaft 104 (and/or conductive elements 108) should be inserted further or withdrawn. In some embodiments, the indicator comprises one or more circumferential markings (such as one or more colored bands) printed on the second shaft 104.

When the conductive elements 108 are in a desired position, stimulation energy is delivered to the treatment site. In some embodiments, the delivered current is a unipolar square wave having an amplitude between 1 mA and 50 mA, a frequency of 5 Hz, and a pulse duration of 200 μS. Each treatment session can have a duration between 5 minutes and 20 minutes. For example, the treatment session can have a duration of 10 minutes. In some embodiments, a patient can undergo a single treatment per day over the course of multiple days of treatment. For example, a patient can undergo one treatment session per day for three to six consecutive days. In some embodiments, the patient may undergo multiple treatment sessions per day and/or per week. Still, other current parameters and treatment parameters are possible.

### III. Intraluminal Stimulation to Rehabilitate Speech

Traumatic brain injury causes damage to the brain that can result in speech and language problems. The speech produced by a person who has traumatic brain injury may be slow, slurred, and difficult or impossible to understand if the areas of the brain that control the muscles of the speech mechanism are damaged. This type of speech problem is called dysarthria. Others may have what is called apraxia of speech, a condition in which strength and coordination of the speech muscles are unimpaired but the individual experiences difficulty saying words correctly in a consistent way. Prolonged disuse due to extended periods of mechanical ventilation can also have the foregoing effects. The presence of an oral intubation tube or a cuffed tracheostomy tube prevents the flow of air through the vocal cords required to create the sounds associated with speech. Muscles responsible for moving laryngeal structures can become atrophied or even physically damaged by the presence of the breathing tube.

The present technology includes a device for delivering an electrical stimulus to nerves proximate a pharynx of a human patient to rehabilitate a patient's speech. As used herein, “rehabilitate” can mean complete or partial rehabilitation, and can include improvements in the physical performance of speech-related function and/or improvements in at least one of sound formation, quality of tone, intelligibility of communication, or level of speech. In some embodiments, the device includes an elongated member having a proximal portion configured to be coupled to an energy source and a distal portion configured to be positioned at a treatment site within a portion of the patient's upper gastrointestinal tract, such as the pharynx, the esophagus, or both. The device may further comprise one or more conductive elements carried by the distal portion of the elongated member and configured to be electrically coupled to the energy source to deliver an electrical stimulus to nerves at or proximate the pharyngeal wall at the treatment site. In some embodiments, the conductive elements may be configured to deliver an electrical stimulus to sensory nerves at or proximate one or more laryngeal structures, such as the posterior cricoarytenoid, cricothyroid or vocalis. The electrical stimulation of the present technology is configured to excite central nervous system regions associated with speech control and modulation. The electrical stimulus is configured to rehabilitate a patient's speech within a single treatment session and/or over multiple treatment sessions. The device, for example, may comprise any of the treatment systems 10 and/or stimulation devices 100 disclosed herein. In some embodiments, the stimulation device for rehabilitating a patient's speech includes a feeding tube, and in some embodiments the stimulation device does not include a feeding tube.

A method for rehabilitating a patient's speech can include positioning a conductive element of a stimulation device through a nasal or oral cavity and into a lumen of an upper gastrointestinal tract of a patient, and delivering stimulation energy to sensory nerves proximate the lumen. In some embodiments, the stimulation energy has a frequency of about 5 Hz to about 80 Hz, an amplitude of about 1 mA to about 50 mA, and/or a pulse width of about 200 μS to about 1 mS.

The sensory nerves receiving the electrical stimulus, for example, can be associated with speech. As used herein, the terms “speech-related nerves” or “nerves associated with speech” refer to the nerve or a muscle for which normal function includes activity that affects, or contributes to affecting, all or any part of normal speech function. Over the course of one or several treatments, the stimulation energy can improve one or both of the patient's speech resonance and phonation. In some embodiments, the sensory nerves include one or more cranial nerves, such as one or more branches of the glossopharyngeal nerve and/or one or more branches of the vagus nerve. The stimulation energy can be delivered while the conductive element is positioned within the patient's pharynx, a portion of the oropharynx, and/or a portion of the laryngopharynx. In some embodiments, the stimulation is delivered while the conductive element is in apposition with a posterior wall of the pharynx, such as the posterior pharyngeal wall along the oropharynx, and/or the posterior pharyngeal wall along the laryngopharynx. In some embodiments, the stimulation device and/or conductive element(s) may be positioned in apposition with a lateral, medial, and/or anterior portion of the pharyngeal wall and/or not the posterior portion. In those embodiments where the device includes a feeding tube, the feeding tube may remain positioned in the patient's upper gastrointestinal tract when the stimulation energy is delivered.

Each treatment session can have a duration between 5 minutes and 20 minutes. For example, the treatment session can have a duration of 10 minutes. In some embodiments, a patient can undergo a single treatment per day over the course of multiple days of treatment. For example, a patient can undergo one treatment session per day for three to six consecutive days. In some embodiments, the patient may undergo multiple treatment sessions per day and/or per week. Still, other current parameters and treatment parameters are possible.

### IV. Intraluminal Stimulation to Improve Gastric Motility

The estimated annual healthcare expenditures for gastrointestinal (GI) motility disorders is USD 29 billion, which imposes a significant burden on the U.S. healthcare system. GI motility disorder is defined as the abnormal movement behavior of the GI tract that affects the function of mixing and propelling food. This can happen to any segment of the GI tract, including the esophagus (e.g., dysphagia, achalasia), stomach (e.g., gastroparesis, gastroesophageal reflux disease), and intestines (e.g., diarrhea, constipation). The pathophysiological mechanism of GI motility disorders is not completely understood due to the complex, cooperative mechanisms between the smooth muscle cells (SMC), interstitial cells of Cajal (ICC), enteric nervous system, central nervous system, and hormones. This gap of knowledge impedes the development of efficacious therapies for improving GI motility. Although a number of pharmaceutical drugs have been developed, most of them do not completely alleviate GI dysmotility. Moreover, the use of drugs faces the challenge of target specificity, as it is difficult to precisely control the dose of pharmacological agents.

Damage to the brain caused by traumatic brain injury or prolonged disuses can result in gastric motility disorders.

The present technology includes a device for delivering an electrical stimulus to nerves proximate a pharynx and/or esophagus of a human patient to treat a gastric motility disorder. In some embodiments, the device includes an elongated member having a proximal portion configured to be coupled to an energy source and a distal portion configured to be positioned at a treatment site within a portion of the patient's upper gastrointestinal tract, such as the pharynx, the esophagus, or both. The device may further comprise one or more conductive elements carried by the distal portion of the elongated member and configured to be electrically coupled to the energy source to deliver an electrical stimulus to nerves at or proximate the pharyngeal wall at the treatment site. In some embodiments, the conductive elements may be configured to deliver an electrical stimulus to nerves at or proximate the esophageal wall. The electrical stimulation of the present technology is configured to excite central nervous system regions associated with gastric motility. The electrical stimulus may be configured to improve at least one of gastric emptying, contractility, or capacity such that gastric motility overall is improved. The electrical stimulus is configured to improve a patient's gastric motility within a single session and/or over multiple sessions. The device, for example, may comprise any of the treatment systems 10 and/or stimulation devices 100 disclosed herein. In some embodiments, the stimulation device for improving a patient's gastric motility includes a feeding tube, and in some embodiments the stimulation device does not include a feeding tube.

A method for improving gastric motility can include positioning a conductive element of a stimulation device through a nasal or oral cavity and into a lumen of an upper gastrointestinal tract of a patient (such as the pharynx and/or esophagus), and delivering stimulation energy to sensory nerves proximate the lumen. The sensory nerves, for example, can be associated with gastric motility. As used herein, the term “nerves associated with gastric motility” refers to the nerve or a muscle for which normal function includes activity that affects, or contributes to affecting, all or any part of normal gastric motility. In some embodiments, the stimulation energy has a frequency of about 5 Hz to about 100 Hz, an amplitude of about 1 mA to about 50 mA, and/or a pulse width of about 50 μS to about 100 μS.

In some embodiments, the sensory nerves include one or more cranial nerves, such as one or more branches of the vagus nerve. The stimulation energy can be delivered while the conductive element is positioned within the patient's pharynx and/or esophagus, such as a portion of the oropharynx and/or a portion of the laryngopharynx. In some embodiments, the stimulation is delivered while the conductive element is in apposition with a posterior wall of the pharynx, such as the posterior pharyngeal wall along the oropharynx and/or the posterior pharyngeal wall along the laryngopharynx. In some embodiments, the stimulation device and/or conductive element(s) may be positioned in apposition with a lateral, medial, posterior, and/or anterior portion of the esophageal wall. In those embodiments where the device includes a feeding tube, the feeding tube may remain positioned in the patient's upper gastrointestinal tract when the stimulation energy is delivered.

Each treatment session can have a duration between 5 minutes and 20 minutes. For example, the treatment session can have a duration of 10 minutes. In some embodiments, a patient can undergo a single treatment per day over the course of multiple days of treatment. For example, a patient can undergo one treatment session per day for three to six consecutive days. In some embodiments, the patient may undergo multiple treatment sessions per day and/or per week. Still, other current parameters and treatment parameters are possible.

### CONCLUSION

Although many of the embodiments are described above with respect to systems, devices, and methods for electrically stimulating a pharynx of a patient, the technology is applicable to other applications and/or other approaches. For example, the device may be used to treat other conditions, or used to apply a different form of energy (such as ablation energy). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

As used herein, the terms “generally,” “substantially,” “about,” and similar terms are used as terms of approximation and not as terms of degree, and are intended to account for the inherent variations in measured or calculated values that would be recognized by those of ordinary skill in the art.

I/We claim:

1. A method for rehabilitating a patient's speech, the method comprising:
via a nasal or oral cavity, positioning a conductive element of a stimulation device in a lumen of an upper gastrointestinal tract of a patient; and
delivering stimulation energy to sensory nerves proximate the lumen, the sensory nerves associated with speech.

2. The method of claim 1, wherein delivering stimulation energy to the sensory nerves excites central nervous system regions associated with speech control and modulation.

3. The method of claim 1, wherein the sensory nerves include at least one of one or more cranial nerves, the glossopharyngeal nerve, or the vagus nerve.

4. The method of claim 1, wherein the sensory nerves include sensory nerves proximate one or more structure of the larynx.

5. The method of claim 1, wherein delivering the stimulation energy improves at least one of sound formation, quality of tone, intelligibility of communication, or level of speech.

6. The method of claim 1, wherein the stimulation energy is delivered while the conductive element is positioned within the pharynx.

7. The method of claim 1, wherein the stimulation energy is delivered while the conductive element is positioned within the laryngopharynx.

8. The method of claim 1, wherein the stimulation energy is delivered while the conductive element is in apposition with a posterior wall of the pharynx.

9. The method of claim 1, wherein the stimulation energy is delivered while the conductive element is in apposition with at least one of an anterior wall of the pharynx, a lateral wall of the pharynx, or a medial wall of the pharynx.

10. The method of claim 1, wherein the stimulation device includes a feeding tube, and wherein the feeding tube remains positioned in the patient's upper gastrointestinal tract when the stimulation energy is delivered.

11. The method of claim 1, wherein the stimulation energy has a frequency of about 5 Hz to about 80 Hz.

12. The method of claim 1, wherein the stimulation energy has an amplitude of about 1 mA to about 50 mA.

13. The method of claim 1, wherein the stimulation energy has a pulse width of about 200 μS to about 1 mS.

14. A device for delivering an electrical stimulus to nerves proximate a pharynx of a human patient, the device comprising:
an elongated member having a proximal portion configured to be coupled to an energy source and a distal portion configured to be positioned at a treatment site within the pharynx; and
a conductive element carried by the distal portion of the elongated member and configured to be electrically coupled to the energy source to deliver an electrical stimulus to nerves at or proximate the pharyngeal wall at the treatment site, wherein the electrical stimulus is configured to improve the physical performance of speech related function.

15. The device of claim 14, wherein the electrical stimulus is configured to improve at least one of speech intelligibility, tone, or level.

16. The device of claim 14, wherein the nerves are sensory nerves.

17. The device of claim 14, wherein the nerves comprise at least one of one or more cranial nerves, glossopharyngeal nerves, or vagus nerves.

18. The device of claim 14, further comprising an elongated shaft configured to be slidably positioned through a lumen of the elongated member.

19. The device of claim 18, further comprising a retaining structure configured to releasably engage the elongated shaft to fix a position of the elongated shaft relative to the elongated member.

20. The device of claim 18, wherein the elongated shaft has a proximal portion and a distal portion configured to be positioned within the patient's stomach, and wherein the elongated shaft is configured to deliver nutrients to the stomach.
